(19) United States
(12) Patent Application Publication
(10) Pub. No.: US 2010/0298566 A1
(43) Pub. Date: Nov. 25, 2010

(54) PROCESS FOR THE PREPARATION OF PALIPERIDONE AND ITS INTERMEDIATES

(75) Inventors: Arulmoli Thangavel, Tamilnadu (IN); Arunagiri Muthulingam, Tamilnadu (IN); Thirugnanasambandan Shanmuganathan, Tamilnadu (IN); Rengasamy Vadivelan, Tamilnadu (IN); Gurusamy Kasiyappan Saravanakumar, Tamilnadu (IN); Rao Siripragada Mahender, Tamilnadu (IN)

Correspondence Address: OLIFF & BERRIDGE, PLC, P.O. BOX 320850, ALEXANDRIA, VA, 22320-4850, US

(73) Assignee: ORCHID CHEMICALS & PHARMACEUTICALS LIMITED, NUNGAMBAKKAM, TAMILNADU (IN)

(21) Appl. No.: 12/734,440

(22) PCT Filed: Nov. 7, 2008
(86) PCT No.: PCT/IB2008/002985

§ 371 Date: Jun. 11, 2010

(30) Foreign Application Priority Data
Nov. 7, 2007 (IN) 2558/CHE2007
Jul. 10, 2008 (IN) 1673/CHE/2008

(51) Int. Cl. C07D 471/04

(52) U.S. Cl. 544/282

(57) ABSTRACT

A process for the preparation of a psychotropic agent Paliperidone. Preferably, this invention relates to a method for the purification of Paliperidone by making its acid addition salts.

### FIELD OF THE INVENTION

The present invention relates to an improved process for the preparation of a Paliperidone and its intermediates. Preferably, this invention relates to an improved process for the preparation of pure Paliperidone by making its acid addition salts.

### BACKGROUND OF THE INVENTION

Paliperidone, 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one, is a 5-HT antagonist and psychotropic agent belonging to the chemical class of benzisoxazole derivatives. It is a racemic mixture having the following structural formula I:

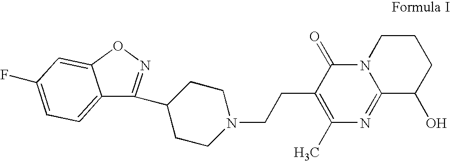

Paliperidone is a metabolite of Risperidone and marketed under the brand name INVEGA®. It is approved in United States for the treatment of schizophrenia.

U.S. '952 patent disclose a process for condensing 9-hydroxy-3-(2-chloroethyl)-2-methyl-6,7,8,9-tetrahydro-4H-pyrido[1,2-α]pyrimidin-4-one of formula (II) with 6-fluoro-3-piperidin-4-yl-benzo[d]isoxazole hydrochloride in a solvent such as methanol and in presence of base such as triethylamine to get paliperidone.

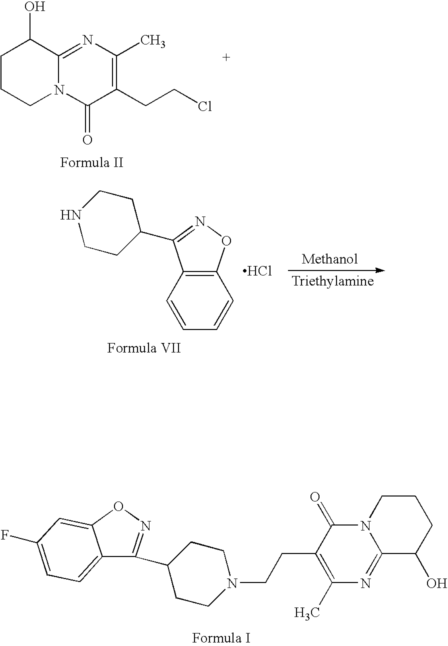

9-hydroxy-3-(2-chloroethyl)-2-methyl-6,7,8,9-tetrahydro-4H-pyrido[1,2-a]pyrimidin-4-one of formula (II), of which is a key intermediate of Paliperidone known from EP-0,368,388 and its U.S. Pat. No. 5,158,952 (henceforth '952) and has following structural formula:

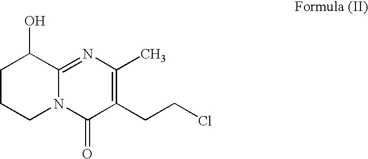

According to the generic disclosure of '952 patent preparation of a compound of formula (II) by catalytic hydrogenation (palladium-on-charcoal and the like) of compound of formula (III) to obtain a compound of formula (IV) which is further converted into a compound of formula (II) by deprotection of benzyl group as mentioned in scheme I

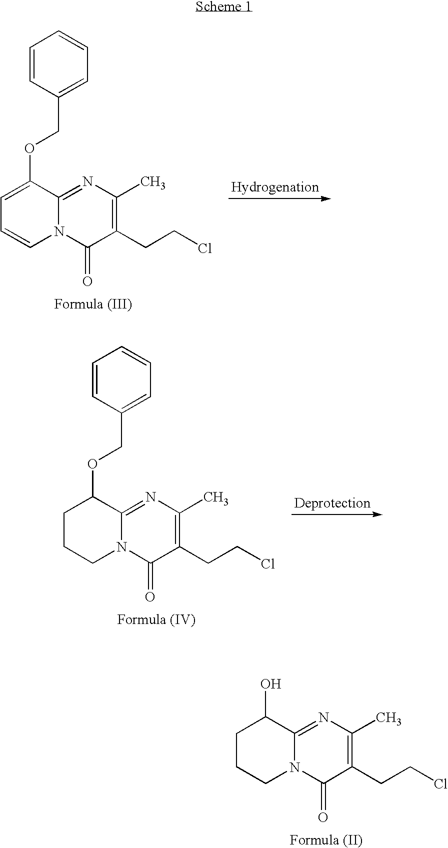

or the catalytic hydrogenation (palladium-on-charcoal and the like) and the deprotection of benzyl group in single step as mentioned in the scheme II

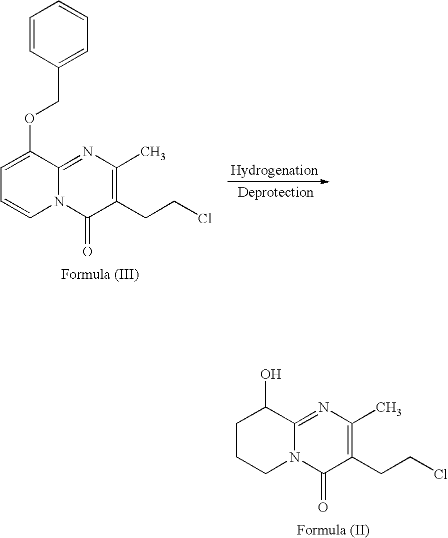

The compound of formula III obtained by condensing 3-benzyloxy-pyridin-2yl-amine of formula (V) with 2-acetyl-3-butyrolactone in an inert solvent to obtain 9-benzyloxy-3-(2-hydroxyethyl)-2-methyl-pyrido[1,2-a]pyrimidin-4one of compound formula IV, which is further reacting with halogenating agent to obtain 9-benzyloxy-3-(2-chloroethyl)-2-methyl-pyrido[1,2-a]pyrimidin-4-one of formula III as mentioned in scheme III

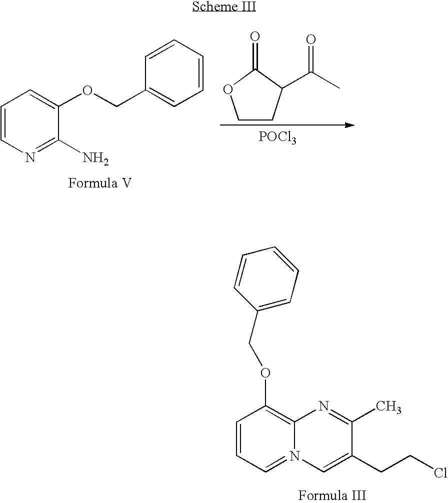

U.S. '952 patent disclose that the staring materials are alternatively prepared by process given in the EP-A-0,196,132.

WO9623784 and WO2006027370 disclose a process for the preparation of 9-hydroxy-3-(2-hydroxyethyl)-2-methyl-4H-pyrido[1,2-a]pyrimidin-4-one by reacting 3-hydroxy-pyridin-2yl-amine with 2-acetyl-3-butyrolactone in an inert solvent and in presence of p-toluene sulfonic acid using water separator.

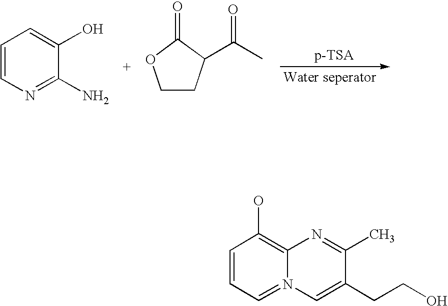

WO2008024415 disclose a process for preparation of 9-hydroxy-3-(2-chloroethyl)-2-methyl-6,7,8,9-tetrahydro-4H-pyrido[1,2-α]pyrimidin-4-one of formula (II) from hydrogenation of compound of formula III to obtain compound of Formula IV followed by removal of benzyl group from compound of Formula IV. This patent advantageously isolating the 9-benzyloxy-3-(2-hydroxyethyl)-2-methyl-pyrido[1,2-a]pyrimidin-4-one of compound formula IV.

The PCT publication WO2008021346 discloses a process for purification paliperidone by slurrying paliperidone in an organic solvent.

As discussed above none of the prior art teaches the synthesis of paliperidone of formula (I) in good purity and hence industrially not viable. Like any synthetic compound, paliperidone can contain extraneous compounds or impurities that can come from many sources. They can be unreacted starting materials, by-products of the reaction, products of side reactions or degradation products. Impurities in paliperidone or any active pharmaceutical ingredient (API) are undesirable and, in extreme cases, might even be harmful to a patient being treated with a dosage form containing the API.

In order to get rid of the toxic impurities, we developed the process for the purification of paliperidone of formula (I) by making its salts

### OBJECTIVE OF THE INVENTION

The main objective of the present invention is to provide an improved process for the preparation of paliperidone of formula (I) in higher purity and greater yield.

Another objective of the present invention is to provide a process for the preparation of compound of formula (III), which would be more simple, economical and easy to implement on commercial scale.

Another objective of the present invention is to provide a process for the preparation of compound of formula (III) in anisole OR diphenyl ether.

In yet another objective of the present invention is to provide acid addition salts of paliperidone.

### SUMMARY OF THE INVENTION

Accordingly, the present invention provides an improved process for the preparation of formula (I) comprising the steps of:

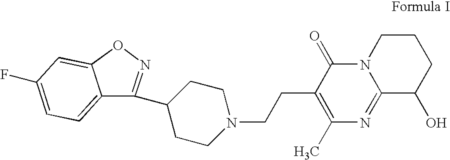

a) condensing 9-hydroxy-3-(2-chloroethyl)-2-methyl-6,7,8,9-tetrahydro-4H-pyrido[1,2-a]pyrimidin-4-one of formula (II)

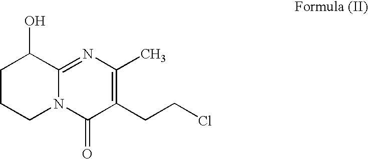

with 6-fluoro-3-piperidin-4-yl-benzo[d]isoxazole or its salt like hydrochloride in a solvent and in presence of base to obtain Paliperidone of formula (I);
b) reacting Paliperidone base of formula (I) with solution of acid in presence of an organic solvent;
c) isolating paliperidone acid addition salt; and
d) converting paliperidone acid addition salt into paliperidone freebase of formula (I).

The above process for the preparation of compound of formula (I) illustrated by following scheme

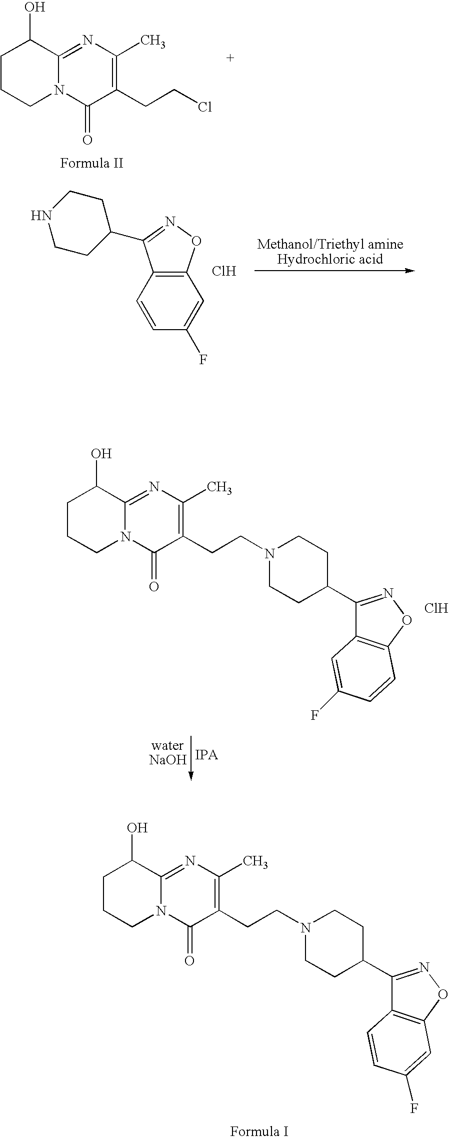

The present invention also provides an improved process for the preparation of compound of formula (III) comprising the steps of:
a) condensing 3-benzyloxy-pyridin-2-yl-amine of formula (V) with 2-acetyl-3-butyrolactone in an inert solvent in presence of dehydration agent and phosphorous oxychloride,
b) isolating 9-benzyloxy-3-(2-hydroxyethyl)-2-methyl-pyrido[1,2-a]pyrimidin-4-one of compound formula IV.

The above process for the preparation of compound of formula (III) illustrated by following scheme

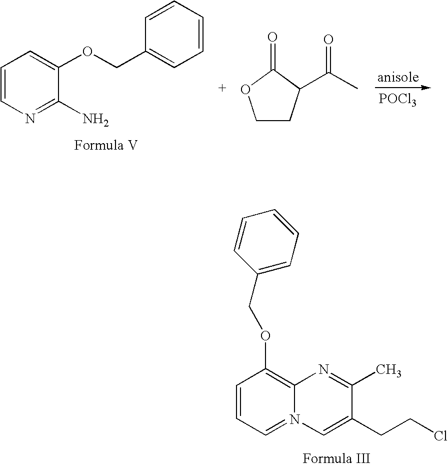

### BRIEF DESCRIPTION OF THE DRAWINGS

The invention will be further described with reference to the following non limiting figures.

FIG. 1 illustrates the X-ray powder diffraction pattern of paliperidone hydrochloride crystalline form;

FIG. 2 illustrates the X-ray powder diffraction pattern of paliperidone hydrobromide crystalline form;

FIG. 3 illustrates the X-ray powder diffraction pattern of paliperidone phosphate crystalline form;

FIG. 4 illustrates the X-ray powder diffraction pattern of paliperidone fumarate crystalline form;

FIG. 5 illustrates the X-ray powder diffraction pattern of paliperidone free base crystalline form obtained by following the innovator process.

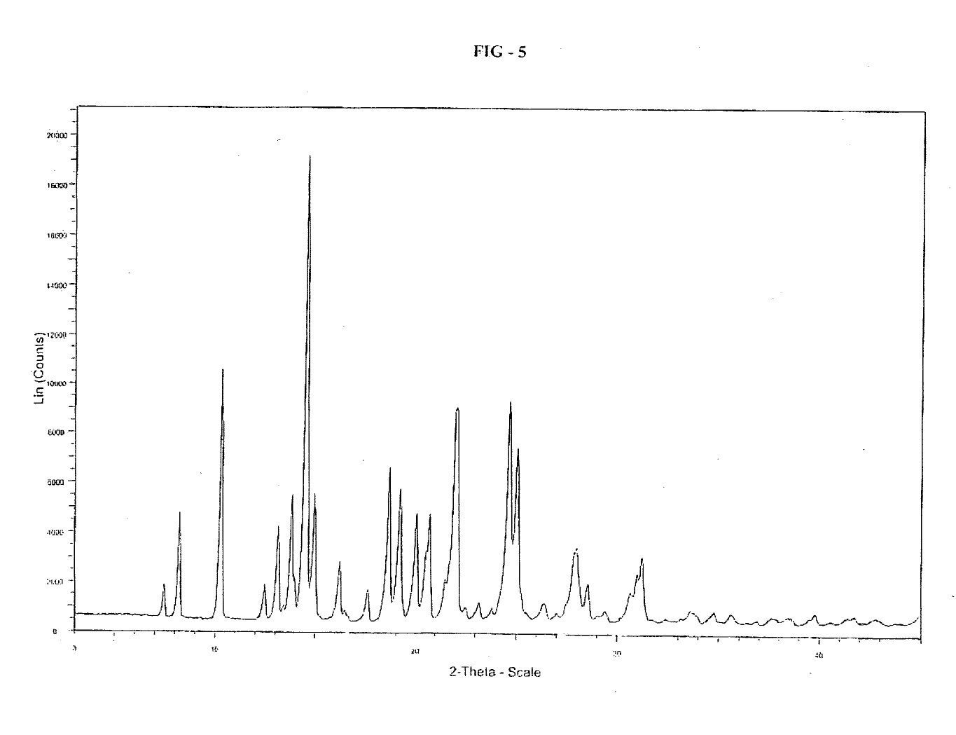

PXRD analyzed by X-Ray Powder Diffractometer of following features:

| | |
|---|---|
| Make | BRUKER AXS |
| Model | D8 ADVANCE |
| Data handling system | EVA 12.0.0.0. |
| ANODE | COPPER |
| RADIATION | COPPER K alpha-1 |
| WAVELENGTH | 1.5406 A° |
| CURRENT & VOLTAGE | 40 kV 30 mA |

### DETAILED DESCRIPTION OF THE INVENTION

In an embodiment of the present invention, the solvent used in step (a) is selected from water, benzene, methylbenzene, dimethylbenzene, chlorobenzene, methoxybenzene, methanol, ethanol, 1-butanol, 2-propanone, 4-methyl-2-pentanone, gamma-butyrolactone, 1,1′-oxybisethane, tetrahydrofuran, 1,4-dioxane, N,N-dimethylformamide, N,N-dimethylacetamide, dimethylsulfoxide, pyridine, 1,3-dimethyl-3,4,5,6-tetrahydro-2(1H)-pyrimidinone, 1,3-dimethyl-2-imidazolidinone, 1,1,3,3-tetramethylurea, 1-methyl-2-pyrrolidinone, nitrobenzene, acetonitrile or a mixture of such solvents.

In another embodiment of the present invention, the base used in step (a) sodium carbonate, sodium hydrogen carbonate, potassium carbonate, sodium hydroxide, calcium oxide, sodium acetate, sodium methoxide, sodium hydride, sodium amide, triethylamine, N-(1-methylethyl)-2-propanamine, 4-ethylmorpholine, 1,4-diazabicyclo[2.2.2]octane, pyridine and the like.

In another embodiment of the present invention, the Paliperidone of formula (I) obtain from step (a) is purified by making its acid addition salts or polymorphic forms of paliperidone acid addition salts. The preparation of Paliperidone by through its acid addition salt found to have advantages, as this process yields final compound in good purity and yield. The acid addition salt of said compound can be isolated by the process or technique known in the prior art either in amorphous form or in crystalline form.

In another embodiment of the present invention, the acid used for making salt of paliperidone is either an organic or inorganic acid, which is selected from hydrochloric acid, hydrobromic acid, hydroiodic acid, o-phosphoric acid, fumaric acid, oxalic acid, and the like; most preferably hydrochloric acid.

In another embodiment of the present invention, where the organic solvent used for the step (b) is selected from group consisting of C₁-C₄ alcohol, C₁-C₄ ketone, halogenated hydrocarbon.

In another embodiment of the present invention, where the organic solvent used for the step (b) is selected from group consisting of methanol, ethanol, isopropanol, acetone, methyl ethyl ketone, dichloromethane, dichloroethane and the like.

In another embodiment of the present invention, the paliperidone acid addition salt is converted into paliperidone free base by reacting acid addition salts or polymorphic forms of paliperidone acid addition salts with alkali solution; where the alkali solution used for the reaction is selected from group consisting sodium hydroxide, sodium carbonate, potassium hydroxide, potassium carbonate preferably sodium hydroxide solution.

In another embodiment of the present invention, acid addition salts of paliperidone which is selected from hydrochloric acid, hydrobromic acid, hydroiodic acid, ortho phosphoric acid, fumaric acid, oxalic acid, and the like.

In another embodiment of the present invention provides crystalline paliperidone hydrochloride characterized by X-ray powder diffraction reflections at about: 9.95, 13.15, 14.54, 17.54, 18.93, 20.51, 23.38, 24.80, 26.54, 28.71, 31.15 and 34.65±0.2 degrees 2θ.

In another embodiment of the present invention provides crystalline paliperidone hydrochloride further characterized by X-ray powder diffraction reflections at about: 7.43, 9.02, 14.81, 17.93, 18.77, 21.63, 23.79, 27.25, 28.31 and 36.11±0.2 degrees 2θ.

In another embodiment of the present invention provides crystalline paliperidone hydrobromide characterized by X-ray powder diffraction reflections at about: 8.41, 10.37, 13.30, 16.81, 18.01, 20.83, 22.71, 25.07, 27.85 and 29.70±0.2 degrees 2θ.

In another embodiment of the present invention provides crystalline paliperidone hydrobromide further characterized by X-ray powder diffraction reflections at about: 5.86, 10.19, 14.38, 17.20, 17.73, 18.76, 19.53, 21.66, 22.94, 24.38 and 28.55±0.2 degrees 2θ.

In another embodiment of the present invention provides crystalline paliperidone phosphate salt characterized by X-ray powder diffraction reflections at about: 7.81, 10.80, 11.40, 12.65, 13.14, 14.22, 16.70, 18.48, 19.23, 20.35, 21.57, 22.27, 24.42, 28.33 and 29.63±0.2 degrees 2θ.

In another embodiment of the present invention provides crystalline paliperidone phosphate salt further characterized by X-ray powder diffraction reflections at about: 6.71, 7.31, 10.29, 12.22, 15.72, 17.23, 19.46, 21.15, 22.83, 25.23, 27.49 28.95 and 32.22±0.2 degrees 2θ.

In another embodiment of the present invention provides crystalline paliperidone fumarate salt characterized by X-ray powder diffraction reflections at about: 7.60, 10.73, 13.81, 15.23, 17.3419.08, 20.27, 22.94, 24.16, 25.83 and 27.64±0.2 degrees 2θ.

In yet another embodiment of the present invention provides crystalline paliperidone fumarate salt further characterized by X-ray powder diffraction reflections at about: 8.21 10.32, 11.44, 14.65, 15.89, 18.52, 21.04, 24.75, 26.98 and 29.11±0.2 degrees 2θ.

In yet another embodiment of the present invention provides an inert solvent used for the preparation of compound of Formula (III) is selected from group anisole, toluene, xylene, diphenyl ether, sulfolane preferably anisole. Surprisingly the use of anisole gives better yield and same was not reported in literature.

In yet another embodiment of the present invention provides dehydrating agent used for the reaction is para-toluenesulphonic acid monohydrate, sulfuric acid, hydrochloric acid preferably para-toluenesulphonic acid monohydrate.

In the present invention the starting materials are prepared according to the literature available in the prior art, or by following the process given in the examples

Advantages of the Process:

Following compounds are major impurities formed by prior art process, which are minimized or removed in present process by precipitating paliperidone as its acid salts including hydrochloric acid, hydrobromic acid, ortho phosphoric acid, fumaric acid, oxalic acid and other organic and inorganic acids.
a) 3-{2-[4-(5-Fluoro-benzo[d]isoxazol-3-yl)-piperidin-1-yl]-ethyl}-2-methyl-7,8-dihydro-6H-pyrido[1,2-a]pyrimidine-4,9-dione
b) 9-Hydroxy-3-(2-chloroethyl)-2-methyl-6,7,8,9-tetrahydro-4H-pyrido (1,2-a) pyrimidine-4-one
c) 6-Fluoro-3-(4-piperidinyl)-1,2-benzisoxazoleHydrochloride
d) 3-{2-[4-(5-Fluoro-benzo[d]isoxazol-3-yl)-piperidin-1-yl]-ethyl}-9-hydroxy-2-methyl-pyrido[1,2-a]Pyrimidin-4-one
e) 3-{2-[4-(5-Fluoro-benzo[d]isoxazol-3-yl)-piperidin-1-yl]-ethyl}-2-methyl-6,7,8,9-tetrahydro-Pyrido[1,2-a]pyrimidin-4-one
f) 3-{2-[4-(5-Fluoro-benzo[d]isoxazol-3-yl)-piperidin-1-yl]-ethyl}-9-hydroxy-2-methyl-6,7,8,9-tetrahydro-pyrido[1,2-a]pyrimidin-4-one
g) 3-(2-{4-[(2,4-Difluoro-phenyl)-hydroxyimino-methyl]-piperidin-1-yl}-ethyl)-9-hydroxy-2-methyl-6,7,8,9-tetrahydro-pyrido[1,2-a]pyrimidin-4-one

The present invention is illustrated with the following example, which should not be construed for limiting the scope of the invention.

### Example (1)

### General Procedure for Preparing Crude Paliperidone Base

To a methanol (500 mL) solution, nitrogen was purged for 30 minutes to remove the nascent oxygen. 6-Fluoro-3-piperidin-4-yl-benzo[d]isoxazole Hydrochloride (50 g), 9-Hydroxy-3-(2-chloroethyl)-2-methyl-6,7,8,9-tetrahydro-4H-pyrido (1,2-a) pyrimidine-4-one (52 g) and triethylamine (55) were added and stirred at reflux for 30-32 hours. The reaction mixture was cooled to 25-35° C. and filtered off to yield paliperidone base.

Yield: 75 g

### Example (2)

### Process for Paliperidone Hydrochloride

To a stirred solution of wet Paliperidone base (5 g) and methanol (50 mL), aqueous hydrochloric acid (33%, 1.0 mol) was added drop wise and allowed to stir at 25-35° C. for 1 hour, followed by stirring at 0-5° C. The reaction mixture was filtered off and washed the solid with methanol. The obtained solid was dried at 50° C. to yield 4.0 g of title compound.

### Example (3)

### Process for Paliperidone Hydrobromide

To a stirred solution of wet Paliperidone base (5 g) and methanol (50 mL), aqueous hydrobromic acid (48%, 1.0 mol) was added drop wise and allowed to stir at 25-35° C. for 1 hour, followed by stirring at 0-5° C. The reaction mixture was filtered off and washed the solid with methanol. The obtained solid was dried at 50° C. to yield 4.2 g of title compound.

### Example (4)

### Process for Paliperidone Phosphate

To a stirred solution of wet Paliperidone base (5 g) and methanol (50 mL), orthophosphoric acid (85%, 1.0 mol) was added drop wise and allowed to stir at 25-35° C. for 1 hour, followed by stirring at 0-5° C. The reaction mixture was filtered off and washed the solid with methanol. The obtained solid was dried at 50° C. to yield 3.6 g of title compound.

### Example (5)

### Process for Paliperidone Fumarate

To a stirred solution of wet Paliperidone base (5 g) and dichloromethane (200 mL), fumaric acid (1.0 mol) in methanol (25 mL) was added drop wise and allowed to stir at 25-35° C. for 1 hour. The reaction mixture is distilled off and the acid salt is isolated in Methanol (50 mL) by stirring it at 0-5° C. The reaction mixture was filtered off and washed the solid with methanol. The obtained solid was dried at 50° C. to yield 4.2 g of title compound.

### Example (6)

### General Procedure for Making Paliperidone from its Corresponding Acid Salt

To a solution of Paliperidone acid salt (5 g) in water (200 mL), the solution of sodium hydroxide (0.45 g, 1.0 mol) in water (10 mL) is added drop wise. The reaction mixture is stirred at 25-35° C. for 1 hour and filtered off. The wet base is slurried in methanol and dried at 50° C. to yield 3.5 g of title compound.

### Example (7)

### Preparation of 9-benzyloxy-3-(2-chloroethyl)-2-methyl-4H-pyrido[1,2-a]pyrimidin-4-one of Formula III

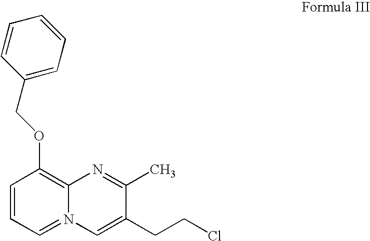

To a solution of 2-acetyl-3-butyrolactone (20 g) in anisole, 2-amino-3-benzyloxy pyridine (25 g) was added and stirred. To the reaction mass, para-toluenesulphonic acid monohydrate was added, stirred and refluxed in a reaction vessel using water separator. Phosphorous oxy chloride (96 g) was added slowly at 25-40° C. to the reaction mixture and stirred for 3-4 hrs at 100-110° C. To the reaction mixture water was added and layers were separated. Sodium hydroxide solution was added to the aqueous layer. The obtained solid was filtered and refluxed with isopropanol. The reaction mass was cooled and the resulting crystal was filtered, washed with isopropanol and dried to obtain of 9-benzyloxy-3-(2-chloroethyl)-2-methyl-4H-pyrido[1,2-a]pyrimidin-4-one.

Yield: 68.7%

### Example (8)

### Preparation of 9-hydroxy-3-(2-chloroethyl)-2-methyl-6,7,8,9-tetrahydro-4H-pyrido[1,2a]pyrimidin-4-one of Formula II

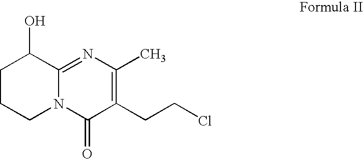

To a 500 mL hydrogenation flask 9-benzyloxy-3-(2-chloroethyl)-2-methyl-4H-pyrido[1,2-a]pyrimidin-4-one (10.0 gm), water (100 mL), Conc. HCl (12 mL) and 10% Pd/C (2.0 gm of 50% wet) were added and the contents were treated with hydrogen gas at pressure 1-2 Kgs under agitation for 4 to 6 hrs at 25 to 30° C. The catalyst was filtered and filtrate was concentrated under vacuum completely to get residue (10 gm). To the residue added water (100 mL) and adjusted the pH to 5-6 with potassium acetate solution (24 gm in 100 mL). The resulting crystal was flittered at 0 to 5° C., washed with water and dried in vacuum to obtain 9-hydroxy-3-(2-chloroethyl)-2-methyl-6,7,8,9-tetrahydro-4H-pyrido[1,2a]pyrimidin-4-one. (5.1 gm, theoretical yield 69%).

1. A process for the preparation of paliperidone of Formula I comprising,
reacting Paliperidone base of formula (I) 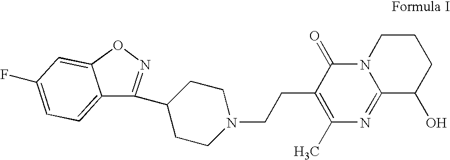
with solution of acid in presence of an organic solvent;
isolating paliperidone acid addition salt; and
converting paliperidone acid addition salt into paliperidone freebase of formula (I).

2. A process according to the claim 1, wherein the acid used for making the salt is an organic or inorganic acid, which is selected from hydrochloric acid, hydrobromic acid, hydroiodic acid, ortho phosphoric acid, fumaric acid, or oxalic acid.

3. A process according to the claim 1, wherein the organic solvent is selected from group methanol, ethanol, isopropanol, acetone, methyl ethyl ketone, dichloromethane, dichloroethane or mixtures thereof.

4. A process according to the claim 1, where the paliperidone acid addition salt is converted into paliperidone free base by reacting acid addition salts with base.

5. A process according to the claim 4, wherein the base is selected from the group consisting of sodium hydroxide, sodium carbonate, potassium hydroxide, and potassium carbonate.

6. Acid addition salts of paliperidone.

7. Paliperidone HCl salt.

8. The acid addition salts of paliperidone of claim 6 is selected from hydrochloric acid, hydrobromic acid, hydroiodic acid, ortho phosphoric acid, fumaric acid, or oxalic acid.

9. Crystalline paliperidone hydrochloride salt characterized by X-ray powder diffraction reflections at about: 9.95, 13.15, 14.54, 17.54, 18.93, 20.51, 23.38, 24.80, 26.54, 28.71, 31.15 and 34.65±0.2 degrees 2θ.

10. Crystalline paliperidone hydrochloride of claim 9 further characterized by X-ray powder diffraction reflections at about: 7.43, 9.02, 14.81, 17.93, 18.77, 21.63, 23.79, 27.25, 28.31 and 36.11±0.2 degrees 2θ.

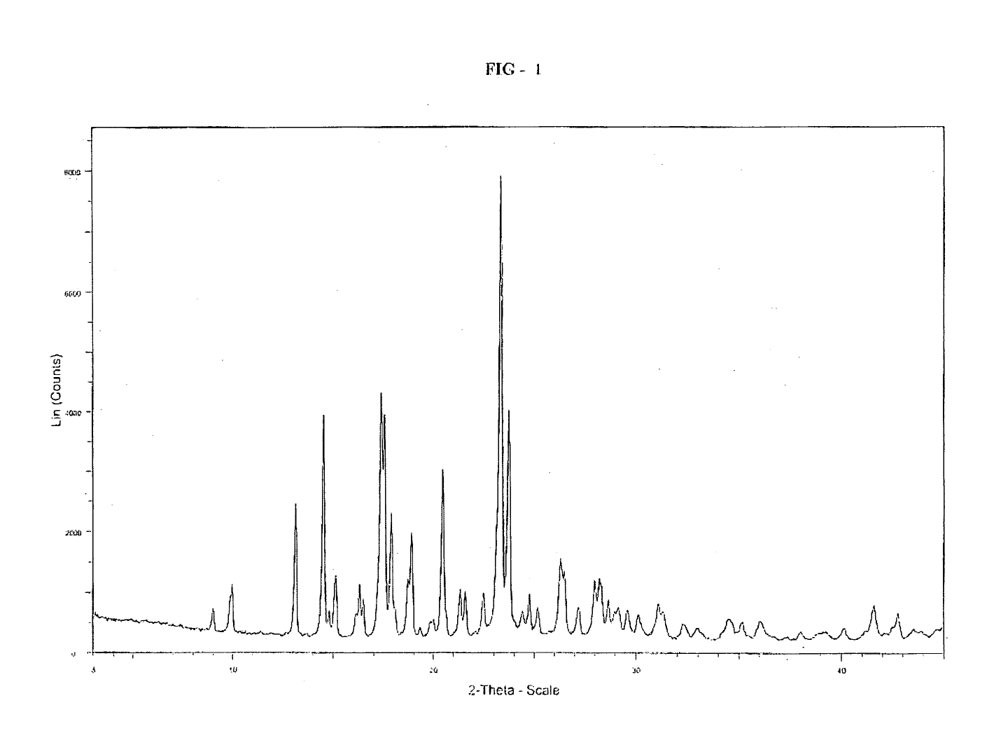

11. Crystalline paliperidone hydrochloride salt having substantially same powder X-ray diffraction (PXRD) pattern as shown in FIG. 1.

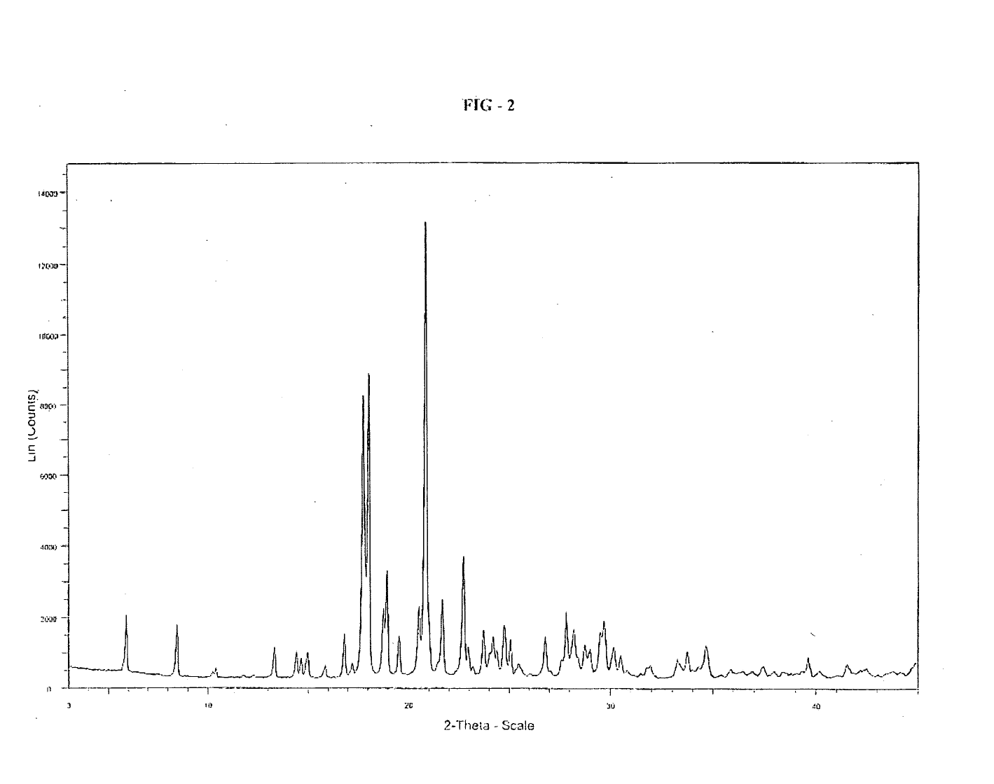

12. Crystalline paliperidone hydrobromide salt having substantially same powder X-ray diffraction (PXRD) pattern as shown in FIG. 2.

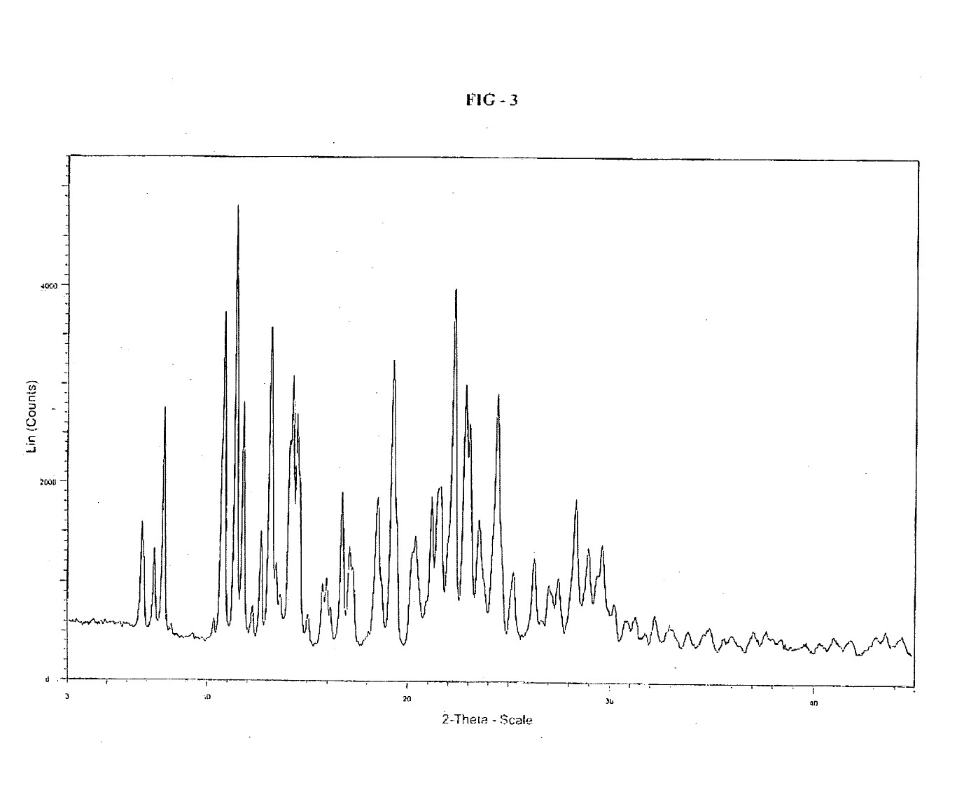

13. Crystalline paliperidone phosphate salt having substantially same powder X-ray diffraction (PXRD) pattern as shown in FIG. 3.

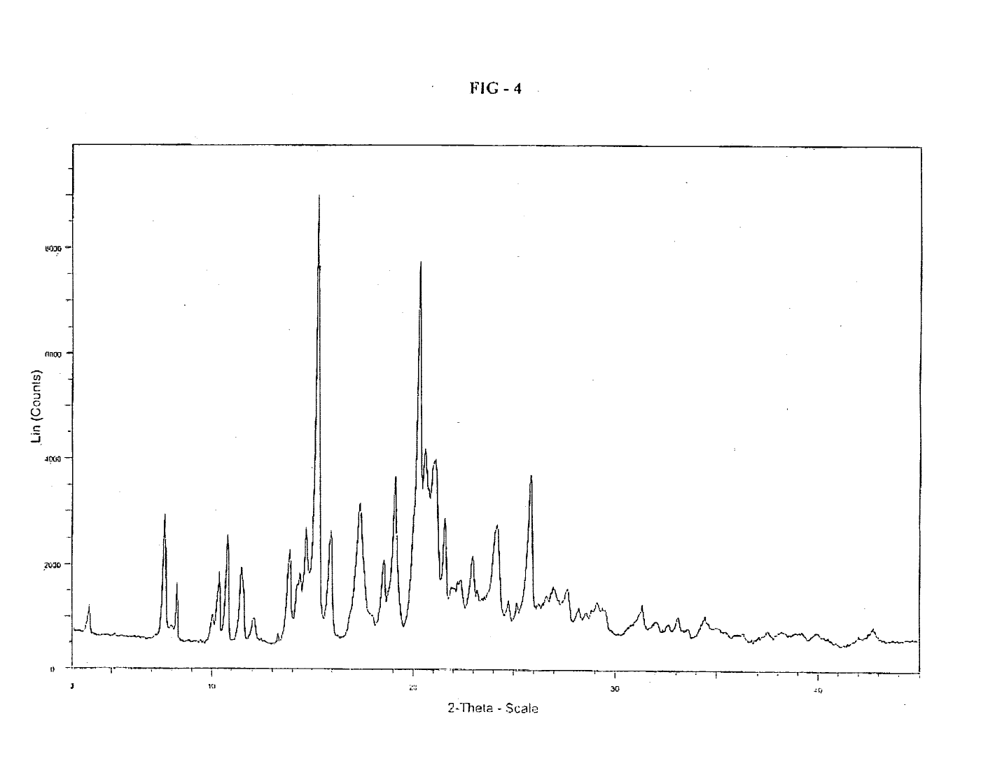

14. Crystalline paliperidone fumarate salt having substantially same powder X-ray diffraction (PXRD) pattern as shown in FIG. 4.

15. A method of preparing a paliperidone base from a crystalline paliperidone acid addition salt.

16. A process for the preparation of 9-benzyloxy-3-(2-chloroethyl)-2-methyl-4H-pyrido[1,2-a]pyrimidin-4-one of compound of formula (III) comprising the steps of: 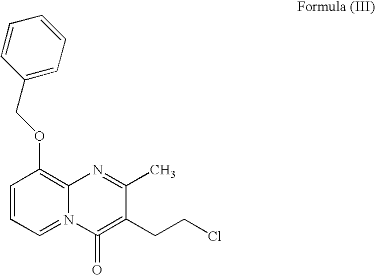
condensing 3-benzyloxy-pyridin-2-yl-amine of formula (V) 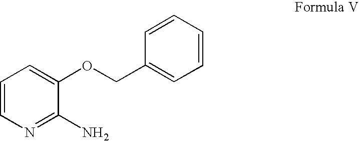
with 2-acetyl-3-butyrolactone in a solvent selected from anisole or diphenyl ether in a presence of dehydration agent and chlorinating agent, and
isolating benzyloxy-3-(2-chloroethyl)-2-methyl-4H-pyrido[1,2-a]pyrimidin-4-one compound of formula (III.

17. A method for preparing a paliperidone of formula (I) 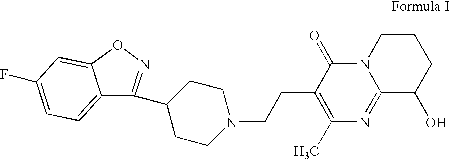
from benzyloxy-3-(2-chloroethyl)-2-methyl-4H-pyrido[1,2-a]pyrimidin-4-one compound of formula (III) prepared by the process according to claim 16.

18. A process for the preparation of Paliperidone of formula (I) comprising, reducing 9-benzyloxy-3-(2-chloroethyl)-2-methyl-pyrido[1,2-a]pyrimidin-4-one compound of formula (III) 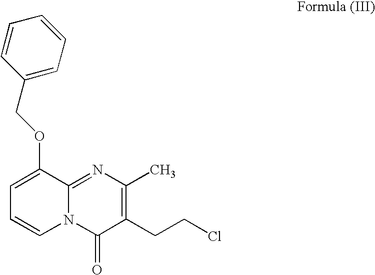
in presence of an aqueous acid;
optionally isolating 9-hydroxy-3-(2-chloroethyl)-2-methyl-6,7,8,9-tetrahydro-4H-pyrido[1,2-α]pyrimidin-4-one of formula (II); 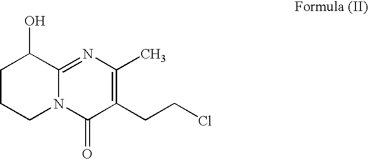
condensing 6-Fluoro-3-piperidin-4-yl-benzo[d]isoxazole of formula (VI) or its acid addition salts to obtain Paliperidone of formula (I); 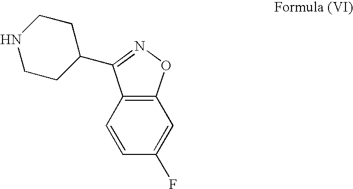
reacting Paliperidone base of formula (I) 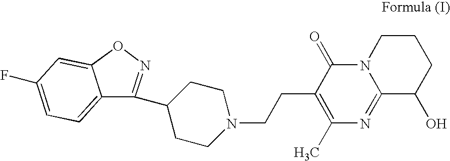
with solution of acid in presence of an organic solvent;
isolating Paliperidone acid addition salt; and
converting Paliperidone acid addition salt into Paliperidone freebase of formula (I).

19. A process for the preparation of compound of formula (II) comprising, 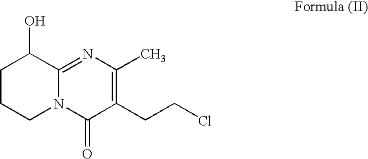
reducing 9-benzyloxy-3-(2-chloroethyl)-2-methyl-pyrido[1,2-a]pyrimidin-4-one compound of formula (III) in presence of an aqueous acid.

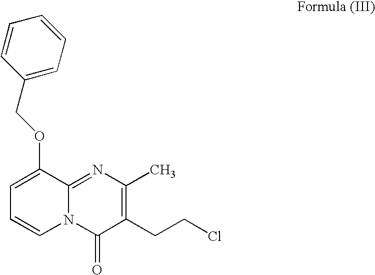

20. The process according to the claim 18, wherein the acid used for reduction is aqueous hydrochloric acid.
